(19) United States
(12) Patent Application Publication
(10) Pub. No.: US 2020/0100780 A1
(43) Pub. Date: Apr. 2, 2020

(54) LAMINATED SURGICAL HANDPIECE AND METHOD FOR FORMING SAME

(71) Applicant: COVIDIEN LP, Mansfield, MA (US)

(72) Inventor: PAUL F. PROKOP, WOBURN, MA (US)

(21) Appl. No.: 16/583,488

(22) Filed: Sep. 26, 2019

Related U.S. Application Data
(60) Provisional application No. 62/737,292, filed on Sep. 27, 2018.

(51) Int. Cl. A61B 17/00; B32B 37/12; B32B 3/26

(52) U.S. Cl. CPC A61B 17/00; B32B 37/12; B32B 2535/00; A61B 2017/00526; A61B 2560/04; B32B 3/266

(57) ABSTRACT

A surgical instrument includes a housing defining a longitudinal axis therealong. The housing includes a plurality of pre-formed sections having a first pre-formed section including one or more openings defined therein and a second, adjacent pre-formed section including one or more openings defined therein in registry with the corresponding one or more openings defined within the first pre-formed section. The one or more openings of the first and second pre-formed sections, when assembled, define a length to each corresponding one or more openings along the longitudinal axis of the housing. An adhesive is configured to bond the first and second pre-formed sections together.

### BACKGROUND

### Technical Field

The present disclosure relates to surgical instruments and, more particularly, to surgical instruments for use with surgical procedures and methods of manufacturing the same.

### Description of Related Art

Surgical instruments come in many shapes and sizes and typically include a variety of parts that are molded or extruded and then assembled to form the surgical instrument. Many of these instruments require multifaceted geometries and contours which adds to the complexity of the instrument especially when interior cavities or through-holes must be formed to accommodate various parts, ingress ports, exhaust ports, or electrical circuitry and wiring. Other instruments are cast or machined which may lead to additional manufacturing difficulties. For example, internal geometries such as holes, chambers or cavities are very difficult to machine or cast.

### SUMMARY

As used herein, the term “distal” refers to the portion that is being described which is further from a user, while the term “proximal” refers to the portion that is being described which is closer to a user.

In accordance with one aspect of the present disclosure, a surgical instrument includes a housing defining a longitudinal axis therealong, the housing including a plurality of pre-formed sections. The plurality of pre-formed sections include a first pre-formed section having one or more openings defined therein and a second, adjacent pre-formed section having one or more openings defined therein in registry with the corresponding one or more openings defined within the first pre-formed section. The one or more openings of the first and second pre-formed sections, when assembled, define a length to each corresponding one or more openings along the longitudinal axis of the housing. An adhesive is configured to bond the first and second pre-formed sections together.

In aspects according to the present disclosure, the first pre-formed section includes an outer periphery that is larger than an outer periphery of the second preformed section. In other aspects according to the present disclosure, one or more of the openings of the first pre-formed section is dimensioned differently than a corresponding and in registry one or more of the openings of the second pre-formed section altering the dimensions of the one or more openings along the length of the housing.

In aspects according to the present disclosure, one or more additional, adjacent pre-formed sections includes at least one less or one more opening defined therein relative to the corresponding and adjacent openings of the first or second pre-formed sections. In other aspects, the first pre-formed section is at least one of machined, die-cast or cut. In still other aspects, the first pre-formed section is made from aluminum, stainless steel, ceramic, plastic, or rubber. In yet other aspects according to the present disclosure, the one or more openings of the first and second pre-formed sections is selected from the group consisting of holes, apertures, chambers and slots.

In accordance with another aspect of the present disclosure, a method of forming a surgical instrument includes: machining, die-casting or cutting a first pre-formed section including one or more openings defined therein; machining, die-casting or cutting a second pre-formed section including one or more openings defined therein; assembling the first and second pre-formed sections to form a housing, the one or more openings in the second pre-formed section in registry with the corresponding openings of the first pre-formed section to define a length to each corresponding one or more openings along a longitudinal axis of the housing; and bonding the first pre-formed section to the second preformed section.

In aspects according to the present disclosure, the method further includes: machining, die-casting or cutting one or more additional, pre-formed sections including at least one less or one more opening defined therein relative to the corresponding and adjacent openings of the first or second pre-formed sections.

In aspects according to the present disclosure, the method further includes: altering a size of one or more openings of the first pre-formed section relative to the size of the corresponding and in registry one or more openings of the second pre-formed section to adjust the size of the at least one opening along the length of the housing.

### BRIEF DESCRIPTION OF THE DRAWINGS

Various aspects of the present disclosure are described herein with reference to the drawings wherein like reference numerals identify similar or identical elements:

FIG. 1 is a side, perspective view of a housing for a surgical instrument according to an aspect of the present disclosure;

FIG. 2A is an exploded, side view of the housing of FIG. 1 including a plurality of pre-formed sections;

FIG. 2B is an enlarged, side view of the housing of FIG. 1;

FIG. 2C is an enlarged, rear offset view of the housing of FIG. 1;

FIG. 2D is an enlarged, rear collapsed view of the housing of FIG. 1; and

FIG. 3 is an enlarged, perspective view of one pre-formed section of the housing of FIG. 1;

### DETAILED DESCRIPTION

Throughout the description, like reference numerals and letters indicate corresponding structure throughout the several views. Also, any particular feature(s) of a particular exemplary embodiment may be equally applied to any other exemplary embodiment(s) of this specification as suitable. In other words, features between the various exemplary embodiments described herein are interchangeable as suitable, and not exclusive.

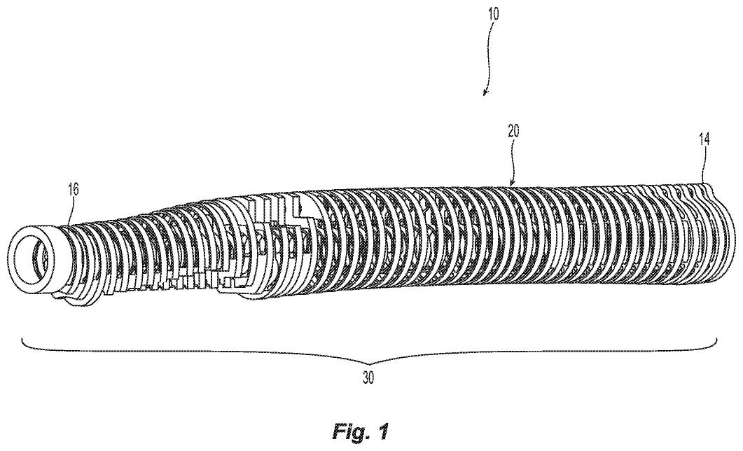

Referring now to FIG. 1, a surgical instrument 10 contemplated for use in connection with traditional open surgical procedures is shown. For the purposes herein, either an open instrument, e.g., instrument 10, or an endoscopic instrument (not shown) may be utilized in accordance with the present disclosure. Obviously, different manufacturing techniques and considerations apply to each particular type of instrument; however, the novel aspects with respect to the surgical instrument 10 remain generally consistent with respect to both the open and endoscopic configurations.

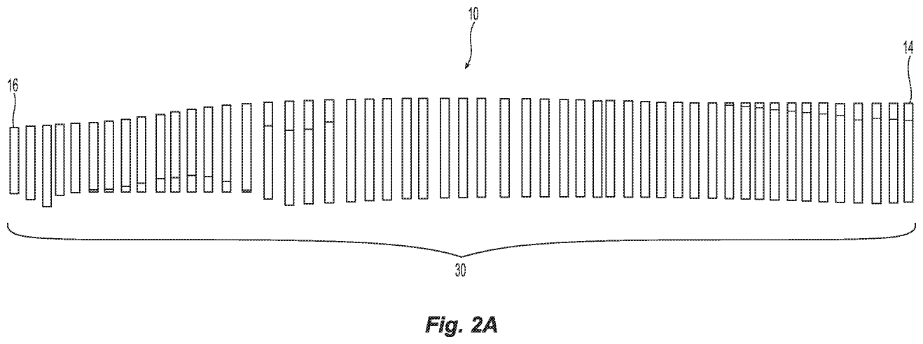

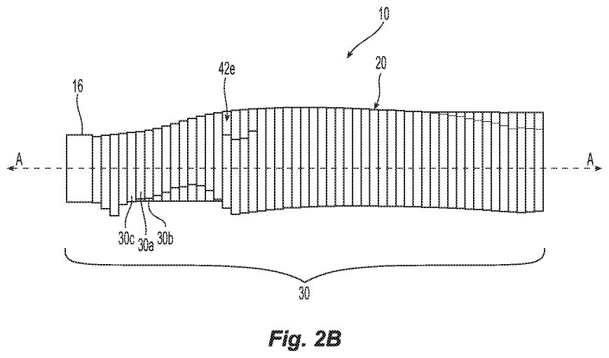

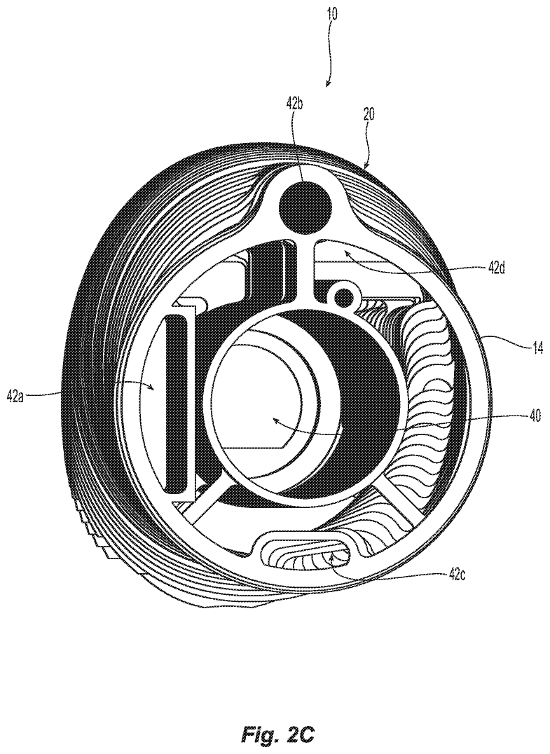

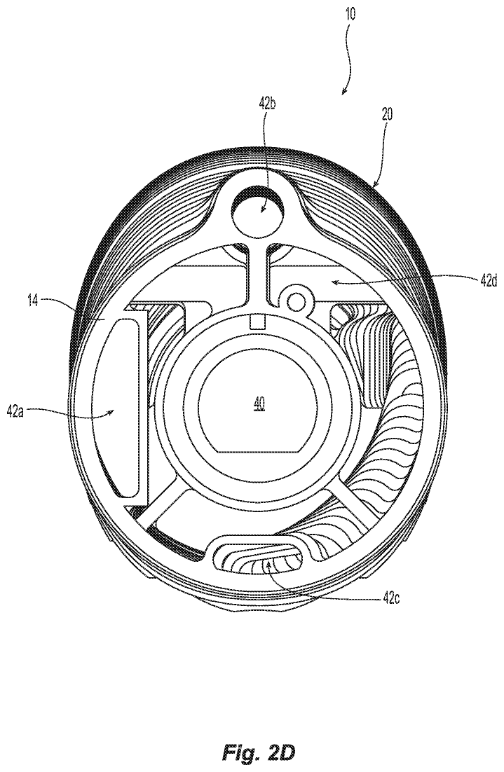

With continued reference to FIG. 1, surgical instrument 10 includes an elongated housing 20 having a proximal end 14 and a distal end 16. Housing 20 includes a plurality of pre-formed sections 30 that, once assembled, form housing 20. Any number of pre-formed sections 30 may be utilized to form housing 20 depending upon a particular surgical purpose, e.g., preformed sections 30, 30b and 30c of FIG. 2B.

Moreover, as explained in more detail below, each pre-formed section 30 may be made from any type of material, e.g., metal sheets (aluminum, stainless steel), ceramic, plastic, rubber, etc. that are joined together by one or more assembly techniques. The pre-formed sections 30 may be machined, die-cast or die-cut and then assembled. For example, each pre-formed section 30 may be cut, cast, or machined from a piece of aluminum clad stock and coated with a brazing material once assembled. Different materials may be joined and utilized depending upon a particular purpose. For example, aluminum pre-formed sections 30 may be used in one part of the housing 20 and ceramic sections (not shown) could be utilized in another part of the housing 20, e.g., to reduce heat conduction. Different techniques may need to be utilized for joining the two different pre-formed sections, e.g., 30a and 30b of FIG. 2B.

FIG. 2A-2D show various views of the assembled pre-formed sections 30. As can be appreciated, each pre-formed section 30 may be sized and dimensioned differently to varying the geometry of the housing 20 along a length thereof. For example, the pre-formed sections 30 disposed proximate the distal end 16 of the housing 20 may be dimensioned smaller that the pre-formed sections 30 disposed proximate the proximal end 14 of the housing 20. Each pre-formed section 30 may be dimensioned to mechanically engage an adjacent proximal pre-formed section 30 and an adjacent distal pre-formed section, e.g., pre-formed section 30a, 30b and 30c. Various mechanical interfaces (not shown) may be employed to facilitate assembly, e.g., detents, grooves, slots, alignment rods, etc.

By machining, cutting or casting the pre-formed sections 30 and then compiling the pre-formed sections 30 during assembly, manufacturer's can design various internal geometries that are difficult to design using traditional manufacturing methods such as machining or casting the entire housing 20. For example, auger-shaped holes, holes that make one or more turns along the housing 20 length, variously-sized chambers or holes, etc. FIGS. 2C and 2D show a variety of holes, slots and chambers 42a-42d that may be formed utilizing this manufacturing technique. Chamber 42a may be defined partially along one side of the housing 20 and elongated slot 42d may be formed on an opposite side thereof and dimensioned to extend along the length of housing 20. A hole 42b may be defined along the top of housing 20 and slot 42c may be disposed opposite hole 42b. A central aperture 40 may be formed along the length of housing 20 in much the same fashion.

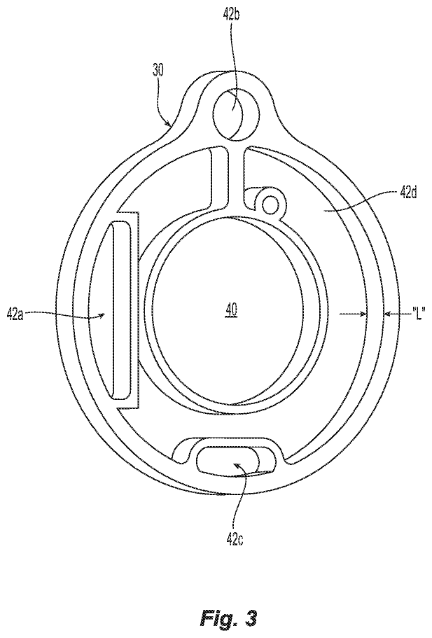

As best shown in FIGS. 2B and 3, each chamber 42a, hole 42b or slot 42c, 42d is cut, die-cast or machined into each pre-formed section 30a and may be dimensioned to extend into the adjacent pre-formed section 30b along the housing 20 or terminate in a given pre-formed section 30c thereby defining the dimensions of each chamber 42a, hole 42b slot 42c, 42d or aperture 40 or initiating a new chamber, hole or slot. Chamber 42a, hole 42b or slots 42c, 42d may be dimensioned to house or support various electrical or mechanical components (not shown), allow the ingress/egress of fluids, or simply provide weight savings to the housing 20.

One or more slots, e.g., slot 42c or holes, e.g., 42b, may be utilized as alignment features prior to laminating or bonding the housing 20 as a final step during the manufacturing process. Slot 42c and/or hole 42b may be configured to receive a rod or key (not shown) to hold and align the plurality of pre-formed sections 30 in place prior to laminating or bonding.

Once the various pre-formed sections 30 are cut, die-cast or machined and assembled, the plurality of pre-formed sections 30 are laminated or bonded together to form housing 20. Lamination is a technique of manufacturing a material in multiple layers, so that the composite material achieves improved strength, stability, sound insulation, appearance or other properties from the use of differing materials. The housing 20 may be permanently assembled and laminated or bonded using heat, pressure, welding, or adhesives.

A method of forming a surgical instrument 10 is also disclosed and includes machining, die-casting or cutting a first pre-formed section 30a including one or more openings 40, 42a, 42b, 42c, and 42d defined therein; machining, die-casting or cutting a second pre-formed section 30b including one or more openings 40, 42a, 42b, 42c, and 42d defined therein; assembling the first and second pre-formed sections 30a, 30b to form a housing 20, the one or more openings 40, 42a, 42b, 42c, and 42d in the second pre-formed section 30b in registry with the corresponding openings 40, 42a, 42b, 42c, and 42d of the first pre-formed section 30a to define a length “L” (FIG. 3) to each corresponding one or more openings 40, 42a, 42b, 42c, and 42d along a longitudinal axis “A” of the housing 20 (FIG. 2B); and bonding the first pre-formed section 30a to the second preformed section 30b.

In aspects according to the present disclosure, the method further includes: machining, die-casting or cutting one or more additional, pre-formed sections 30c (FIG. 2B) including at least one less or one more opening 42e defined therein relative to the corresponding and adjacent openings 40, 42a, 42b, 42c, and 42d of the first or second pre-formed sections 30a, 30b.

In aspects according to the present disclosure, the method further includes: altering a size of one or more openings 40, 42a, 42b, 42c, and 42d of the first pre-formed section 30a relative to the size of the corresponding and in registry one or more openings 40, 42a, 42b, 42c, and 42d of the second pre-formed section 30b to adjust the size of the at least one opening 40, 42a, 42b, 42c, and 42d along the length “L” of the housing 20.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical instrument, comprising:
a housing defining a longitudinal axis therealong, the housing including a plurality of pre-formed sections, the plurality of pre-formed sections including:
a first pre-formed section including one or more openings defined therein;
a second, adjacent pre-formed section including one or more openings defined therein in registry with the corresponding one or more openings defined within the first pre-formed section, wherein the one or more openings of the first and second pre-formed sections when assembled defining a length to each corresponding one or more openings along the longitudinal axis of the housing; and
an adhesive configured to bond the first and second pre-formed sections together.

2. The surgical instrument according to claim 1, wherein the first pre-formed section includes an outer periphery that is larger than an outer periphery of the second preformed section.

3. The surgical instrument according to claim 1, wherein at least one of the one or more openings of the first pre-formed section is dimensioned differently than a corresponding and in registry at least one or more openings of the second pre-formed section altering the dimensions of the at least one or more openings along the length of the housing.

4. The surgical instrument according to claim 1, further comprising at least one additional, adjacent pre-formed section including at least one less or one more opening defined therein relative to the corresponding and adjacent at least one opening of the first or second pre-formed sections.

5. The surgical instrument according to claim 1, wherein the first pre-formed section is at least one of machined, die-cast or cut.

6. The surgical instrument according to claim 1, wherein the first pre-formed section is made from aluminum, stainless steel, ceramic, plastic, or rubber.

7. The surgical instrument according to claim 1, wherein the one or more openings of the first and second pre-formed sections is selected from the group consisting of holes, apertures, chambers and slots.

8. A method of forming a surgical instrument, comprising:
at least one of machining, die-casting or cutting a first pre-formed section including one or more openings defined therein;
at least one of machining, die-casting or cutting a second pre-formed section including one or more openings defined therein;
assembling the first and second pre-formed sections to form a housing, the one or more openings in the second pre-formed section in registry with the corresponding openings of the first pre-formed section to define a length to each corresponding one or more opening along a longitudinal axis of the housing; and
bonding the first pre-formed section to the second preformed section.

9. The method according to claim 8, further comprising:
at least one of machining, die-casting or cutting at least one additional, pre-formed section including at least one less or one more opening defined therein relative to the corresponding and adjacent at least one openings of the first or second pre-formed sections.

10. The method according to claim 8, further comprising:
altering a size of at least one opening of the first pre-formed section relative to the size of the corresponding and in registry at least one opening of the second pre-formed section to adjust the size of the at least one opening along the length of the housing.
